(19) United States
(12) Patent Application Publication
(10) Pub. No.: US 2012/0029452 A1
(43) Pub. Date: Feb. 2, 2012

(54) URINE COLLECTION SYSTEM

(75) Inventor: Carsten Bob Rødsten, Kirke Hyllinge (DK)

(73) Assignee: UROLOGIC APS, Kirke Hyllinge (DK)

(21) Appl. No.: 13/262,122

(22) PCT Filed: Mar. 29, 2010
(86) PCT No.: PCT/DK10/00040

§ 371 Date: Oct. 21, 2011

(30) Foreign Application Priority Data
Mar. 31, 2009 (DK) PA 2009 00432

(51) Int. Cl. A61F 5/453; A61F 5/44

(52) U.S. Cl. 604/353; 604/327; 604/351

(57) ABSTRACT

The present invention relates to a urine collection system for a male user, said collection system comprising a primary urine collection device, wherein the primary urine collection device is adapted to accommodate at least a glans-comprising portion of a penis, said primary urine collection device comprising sealing means adapted to provide an essentially fluid-tight connection between the primary urine collection device and the penis, and wherein said essentially fluid-tight connection comprises a sealing membrane provided with an opening comprising a lip-seal structure sealing against the skin of the penis. According to an embodiment of the invention, a secondary urine collection device is provided, said secondary urine collection device comprising a bladder-like structure adapted to collect body fluids such as urine, and securing means adapted to secure said secondary urine collection device to a leg of the user.

The present invention relates to medical devices, in particular to urine collection systems for male users.

Incontinence and/or having problems due to frequent voiding such as due to LUTS (Lower Urinary Tract Symptoms) can be annoying for a patient. Apart from being tempted to stay in the vicinity of sanitary facilities, incontinence and/or frequent voiding often can lead to social isolation, e.g. when the patient decides to stay at home instead of going to work, and refrains from participating in social events and/or activities.

Incontinence devices/systems usually have to meet one or more requirements, comprising psychological requirements, such as security (against leakage, noise and/or smell), dignity, discretion, prudence etc, as well as mechanical requirements comprising e.g. reliability, convenience, safety, hygiene, ease of application and/or removal, and ease of cleaning and/or disposal. Furthermore, such a device/system should be affordable.

A great number of devices and aids for men suffering from LUTS and incontinence have been on the market for many years and a number of different approaches have been tested. The most simple of these is the urine flask. The flask is a very efficient device in the hospital or at home, but usually not practical in daily use for outpatients. Incontinence devices can be divided into different, sometimes slightly overlapping categories:

I. Absorbents.

Most modern incontinence pads have integrated superabsorbent particles or crystals in order to increase absorbency volume and—as the superabsorbent absorbs the urine and turns into jelly—reduce the wetness of the pad after micturition. Pads for adult incontinent patients are rather big and as they are placed directly in the underpants also rather uncomfortable and indiscrete, especially when wet. Furthermore, the disposal of used pads is often difficult because of the volume and odour.

II. Micturition Bags or Flasks Fixed Directly on Penis.

In general, these systems are usually fixed by a belt around the waist or with a string tied around penis and supported by the underpants. Such designs may possess one or more weaknesses, such as (i) not reliably preventing leakage, (ii) it seems inconvenient to have a urine-container placed in the groin area, (iii) discretion may be at risk, and (iv) other people might hear a splash-noise during micturition.

III. Urine Condoms and Urinals Fixed on Penis, Gland Penis or Scrotum by the Means of Adhesives, Straps, or Friction.

The most widely used device for urine collection on penis is the Urine Condom, also known as Urisheath or Texas condom. Best known are the Conveen and Security+ condoms from Coloplast A/S. Urisheath is a condom with an integrated outlet for connection to a tube. The condom is fixed to penis by means of adhesive or tape providing excellent leakage prevention properties. Alternatively, fixation can be done by the use of a string. Some systems are equipped with vents and/or filters to let air out of the bag and to facilitate a quicker flow from the urisheath into the bag. Application and/or removal of condom-type devices can be uncomfortable and/or even painful, e.g. when pubic hair is trapped and extracted.

US2005101924 and U.S. Pat. No. 4,769,020 concern cups fixed by adhesive tape, while U.S. Pat. No. 4,239,044 pertains to a solution where the collection device is fixed to penis by means of a balloon type system, where inflation of a ring-shaped balloon around penis adds a pressure on the penis skin. In general, inventions relating to fixing a collecting device to the penis gland appear not to have survived on the market.

Inventions using adhesive means applied directly to the penis for fixation have the disadvantage that application can uncomfortable, and removal can even be painful. Furthermore designs relying on applying pressure for fixation are potentially harmful, especially when tightened too much in order to avoid leakage.

IV. Collection Systems Fixated by Means of Underpants, Belts and Straps.

WO2006077351 concerns a urinal with an internal cuff for the penis. The urine is collected in the urinal, which has an outlet in the bottom. The system is placed directly in the underpants and the only fixation is the above-mentioned cuff on the penis. The cuff is for single use.

U.S. Pat. No. 6,010,489 discloses belts and straps to fix a plate around the penis root. The penis is slipped through the plate and a collecting cup is mounted on the plate.

One objective of the invention is to provide an incontinence device and/or system that meets one or more of the herein-mentioned and/or commonly known psychological and/or mechanical requirements in the art, said device or system being based on a mechanical sealing rather than an adhesive sealing.

Another objective of the invention concerns the provision of an affordable solution.

A further objective of the invention deals with providing a device/system that avoids and/or eliminates one or more of the disadvantages of the prior art devices/systems.

Yet a further objective is to provide a re-usable, and userfriendly solution.

Still another objective of the invention relates to providing a solution that avoids and/or prevents irritation and/or tissue damage upon frequent and/or continuous use.

Such objectives are addressed by the present invention.

The present invention provides a urine collection system for a male user comprising a primary urine collection system, and optionally a second urine collection system and connection means adapted to provide a leakage free connection between said primary urine collection device and said secondary urine collection device.

Thus, according to a first aspect of the invention, a secondary urine collection device is provided, said secondary urine collection device being adapted to accommodate body fluids such as urine. According to an embodiment of the invention, the secondary urine collection device comprises a bladder-like structure adapted to collect body fluids, and securing means adapted to releasably secure said secondary urine collection device to a leg of the user.

According to a second aspect of the invention, a primary urine collection device is provided, wherein said primary urine collection device is adapted to accommodate at least a distal portion of a penis, such as the glans-comprising portion, said primary urine collection device comprising sealing means adapted to provide an essentially fluid-tight connection between the first urine collection device and the penis, and wherein said essentially fluid-tight connection comprises a sealing membrane provided with an opening comprising a lip-seal structure sealing against the skin of the penis.

A further aspect of the invention pertains to connection means adapted to provide a leakage-free connection between a primary urine collection device according to the second aspect, and a secondary urine collection device e.g. according to the first aspect, or any such means known per se. A further aspect of the invention relates to diameter determination means adapted to assess a diameter of a body part, such as a penis or a leg. According to one embodiment of the invention, said diameter determination means comprise(s) a ribbon-like structure provided with a symbol- and/or colour-code for simple assessment and/or selection of the appropriate sealing membrane opening, and/or diameter/size of the secondary urine collection device.

According to a further aspect, a kit is provided comprising a primary urine collection device according to the second aspect, a secondary urine collection device according to the first aspect, and optionally connection means e.g. according to one of the further aspects, diameter determination means e.g. according to one of the further aspects, and/or accommodation means e.g. according to one of the further aspects.

A further aspect of the invention relates to accommodation means, such as accommodation means for the primary urine collection device and/or the connection means. According to an embodiment of the invention, such accommodation means may comprise e.g. an opening, such as a slot, hole, or button hole in a garment, such as an underwear, wherein said opening is adapted to provide passage of at least a part or portion of the connection means, such as a tubing or the like from the primary urine collection device to a secondary urine collection device.

According to an embodiment of the invention anti-kinking means are provided, said anti-kinking means being adapted to avoid a reduction and/or blockage of flow of body fluids from the primary urine collection device to e.g. the secondary urine collection device. Such anti-kinking means may comprise the position of the outflow funnel in relation to the sealing membrane, optionally in combination with accommodation means.

Drawings/illustrations are not according to scale, and none of the drawings are to be construed as limiting to the invention. This may also concern any dimension(s) given and/or indicated on any figure and/or drawing.

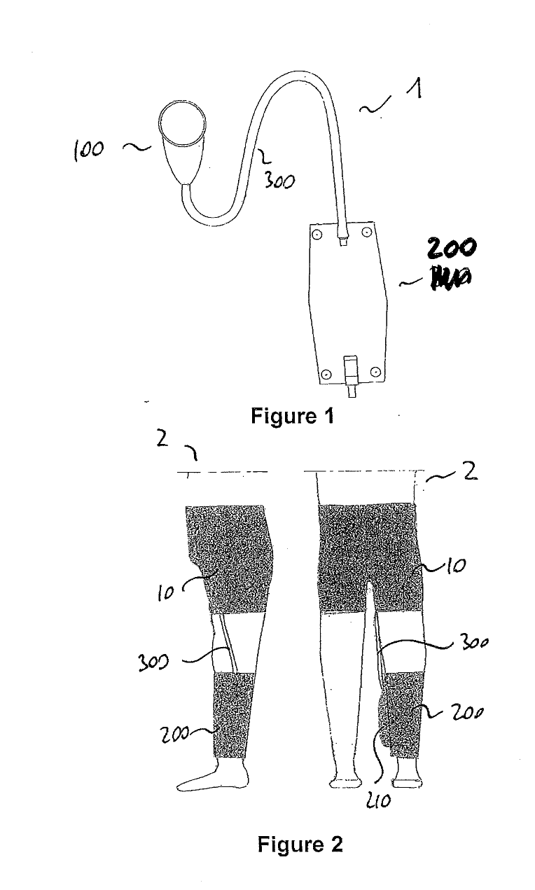

FIG. 1. Schematic drawing of an embodiment of a urine collection system.

FIG. 2. Schematic drawing of another embodiment of a urine collection system in use.

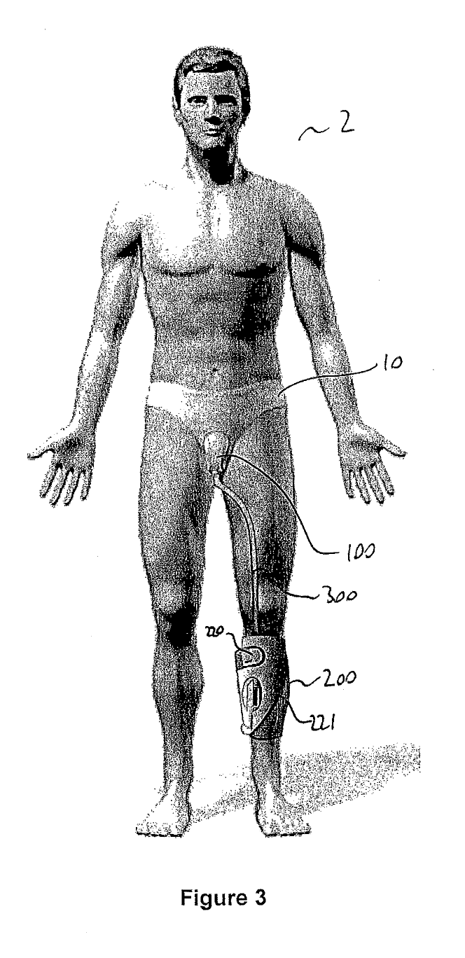

FIG. 3. Schematic illustration of a further embodiment of a urine collection system in use.

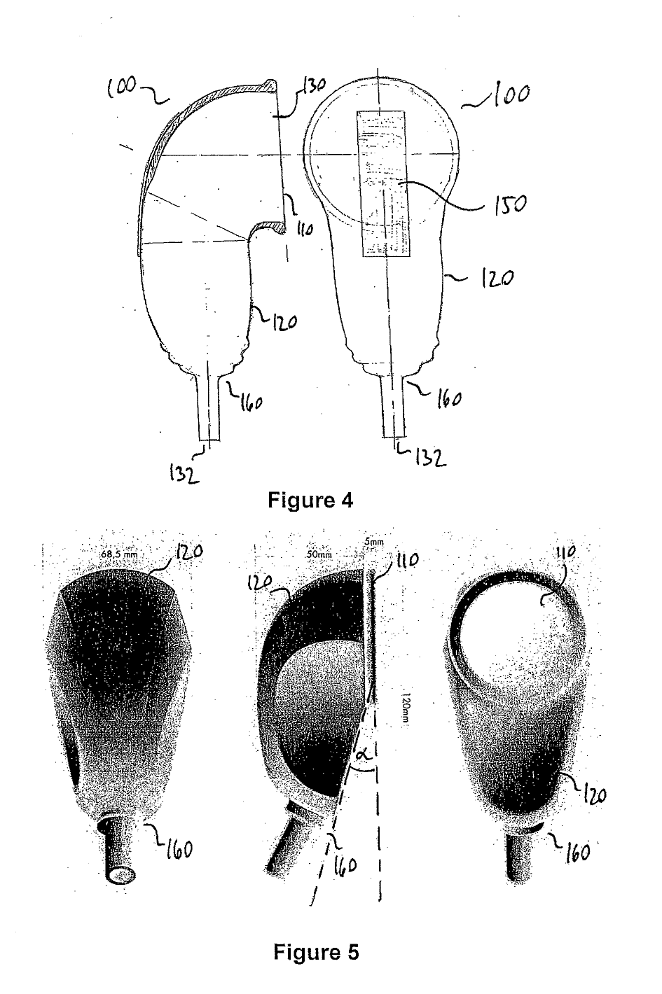

FIG. 4. Schematic drawing of an embodiment of a primary urine collection device.

FIG. 5. Schematic illustration of an embodiment of a primary urine collection device.

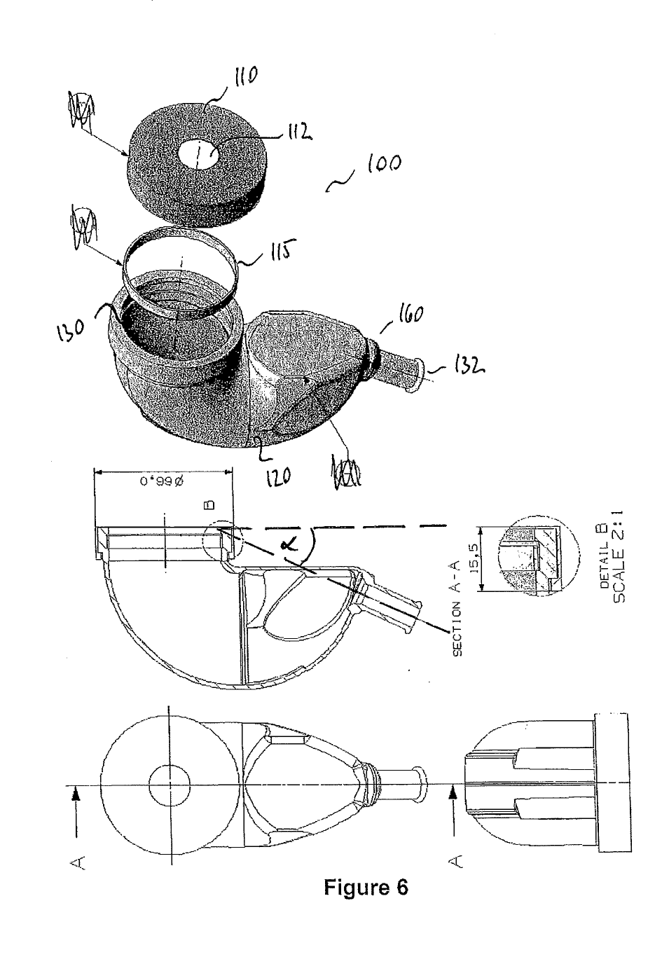

FIG. 6. Schematic drawing of another embodiment of a primary urine collection device.

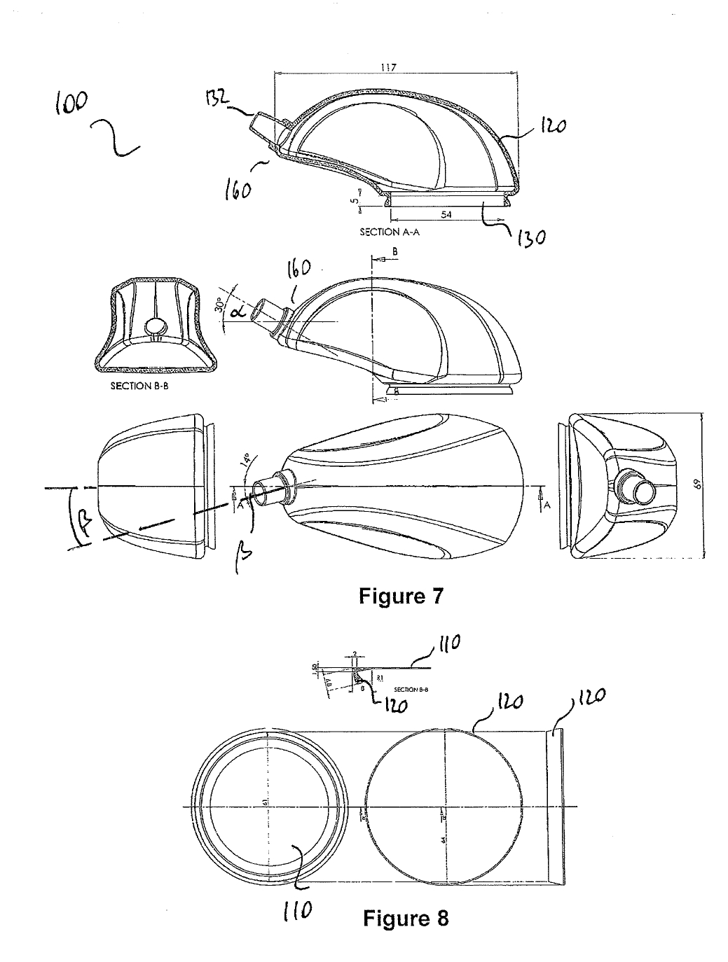

FIG. 7. Schematic drawing of a further embodiment of a primary urine collection device.

FIG. 8. Schematic drawing of an embodiment of a sealing membrane.

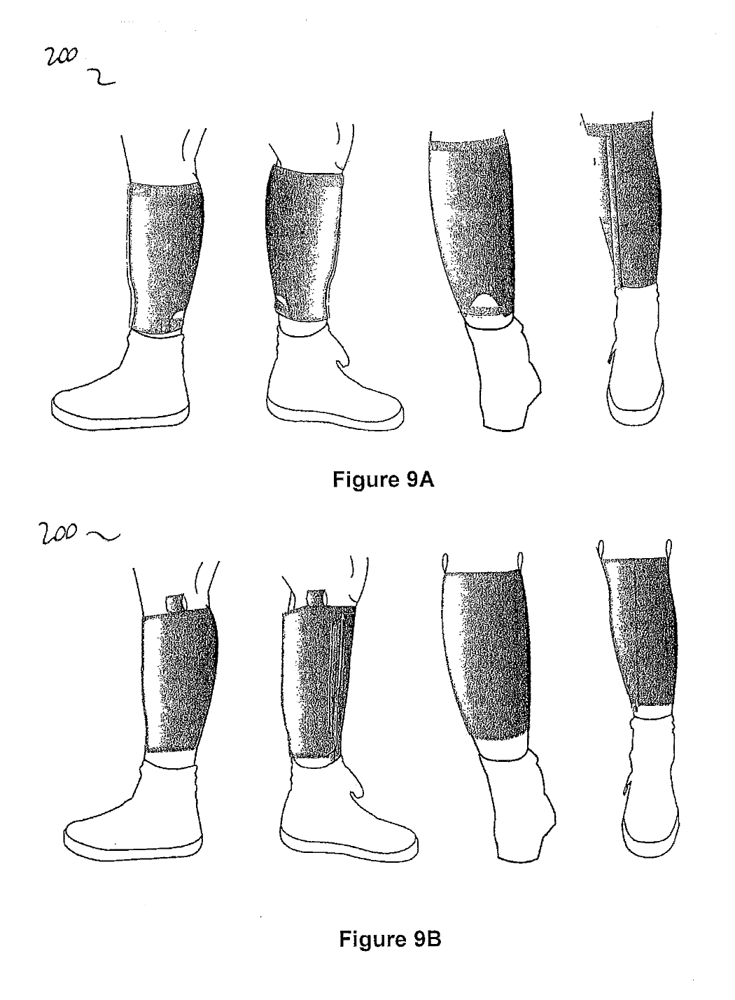

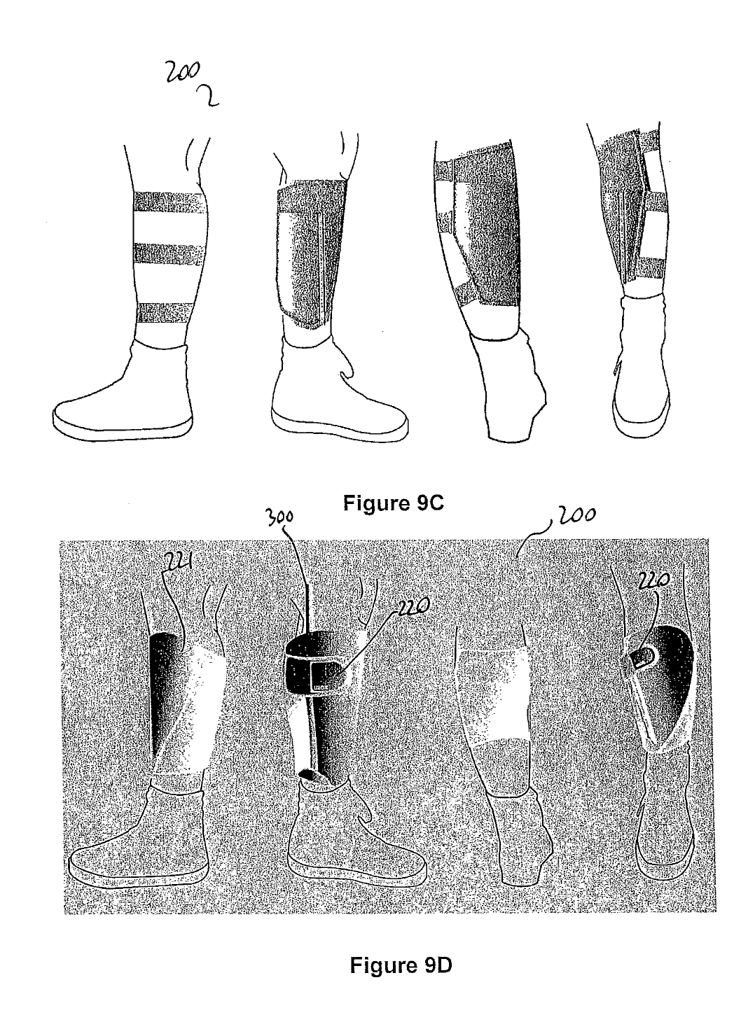

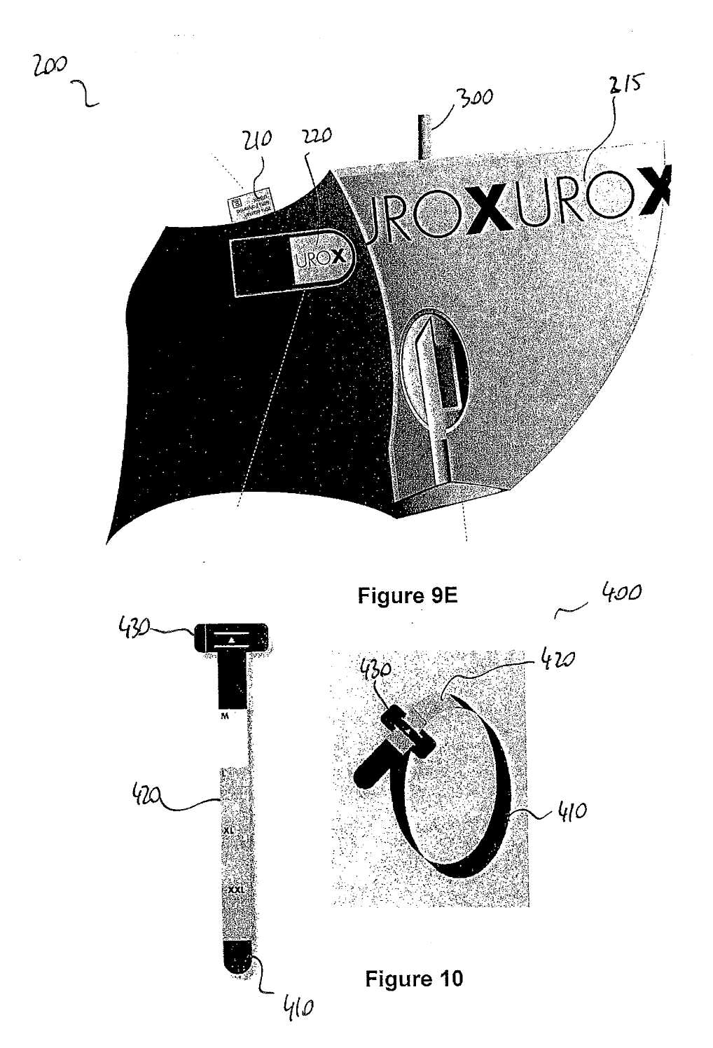

FIG. 9 A-E. Schematic drawing of different embodiments of a secondary urine collection device.

FIG. 10. Schematic drawing of embodiments of diameter determination means.

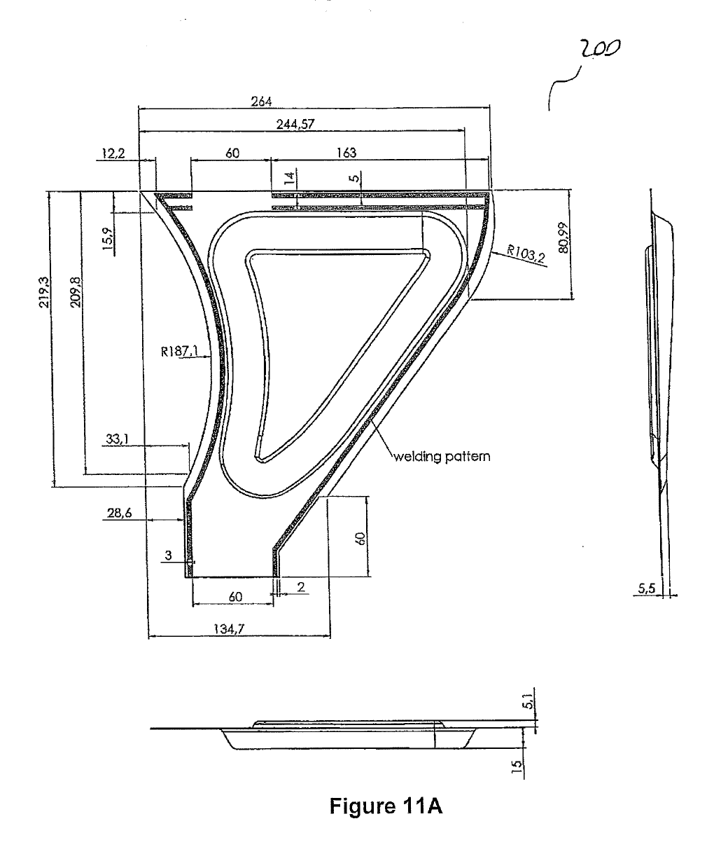

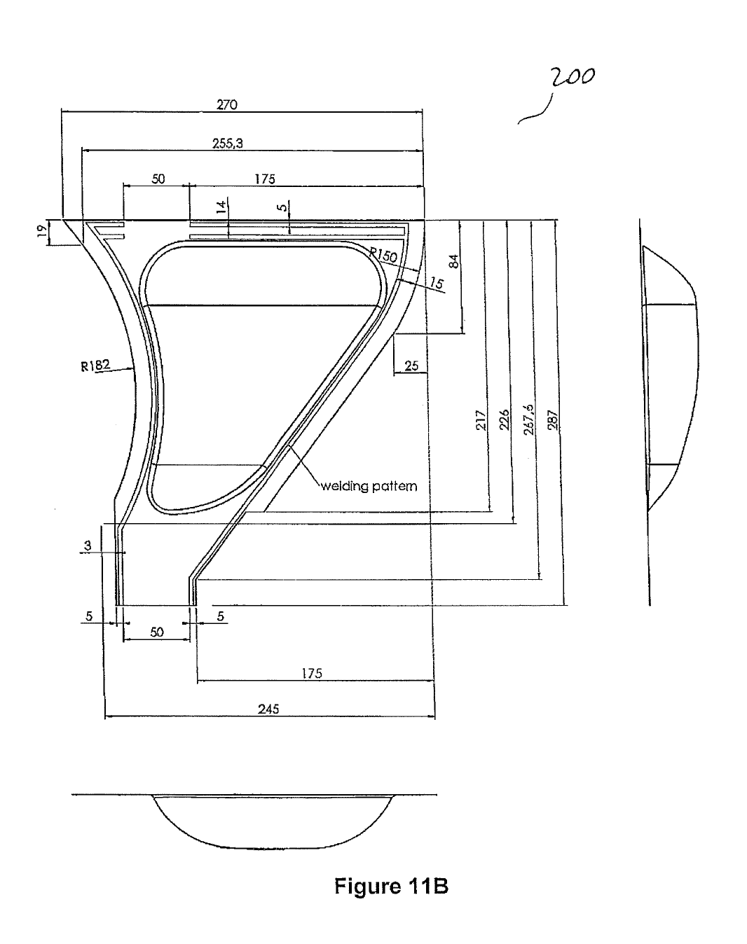

FIG. 11A-B Schematic drawing of further embodiments of a secondary urine collection device.

### DEFINITIONS

About:

The terms “about”, “around”, “approximately”, or “˜” can be used interchangeably. They indicate e.g. the measuring uncertainty commonly experienced in the art, which can be in the order of magnitude of e.g. +/−1, 2, 5, 10, 20, or even 50%.

Comprising:

The term “comprising” or “to comprise” is to be interpreted as specifying the presence of the stated parts, steps, features, or components, but does not exclude the presence of one or more additional parts, steps, features, or components.

User:

In the context of the present invention, the term “user” is meant to comprise the (male) person actually using (i.e. inserting the penis into) the primary urine collection device, also called “primary” user, as well as any other person, e.g. nurse, assistant, etc. assisting said primary user in or during any use-related process of any one or more parts/devices of the urine collection system.

FIG. 1 shows a schematic drawing of an embodiment of a urine collection system comprising a primary urine collection device 100, a secondary urine collection device 200, and connection means 300. In a different embodiment, as shown in FIG. 2, the primary urine collection device 100 is essentially hidden under a garment/underwear 10 the secondary urine collection device 200 is adapted to be secured to a leg of the user and comprises a bladder-like structure 210. The connection means 300 are partially hidden under the garment. In a further embodiment (FIG. 3) the primary urine collection device 100 is essentially not hidden under the garment or underwear 10. The secondary urine collection device 200 is adapted to be secured to the calf of the user by securing means 220, 221.

According to an embodiment of the invention, the primary urine collection device, such as e.g. cup and/or membrane, the secondary urine collection device, the connection means, and/or the size determination means can be re-usable, such as after a quick rinse with water and/or water optionally comprising a cleaning agent. Said primary urine collection device, secondary urine collection device, connection means, and/or the size determination means can also be disinfected and/or sterilized with suitable disinfection and/or sterilizing means known in the art. The secondary urine collection device, and/or parts thereof can be washable, such as suitable for washing in a conventional household washing machine, e.g. using a temperature setting of e.g. 30, 40, or 60° C.

The primary urine collection device can be adapted intermittantly or essentially for continuous use, simple to use, and can be handled/operated by the primary user himself or a secondary user with minimal intrusion of the primary user's privacy.

According to a second aspect of the invention, a primary urine collection device is provided, wherein said primary urine collection device is adapted to accommodate at least a distal portion of a penis. According to an embodiment of the invention, the primary urine collection device is adapted to accommodate at least a glans-comprising portion of a penis, said primary urine collection device comprising sealing means adapted to provide an essentially fluid-tight connection between the first urine collection device and the penis, and wherein said essentially fluid-tight connection comprises a sealing membrane provided with an opening comprising a lip-seal structure sealing against the skin of the penis.

An embodiment of a primary urine collection device (100) is shown in FIG. 4, said device comprising a cup (120) with a first opening (130), a second opening (132), an outflow-funnel (160), and attachment means (150).

According to an embodiment of the invention, the primary urine collection device comprises a cup with a first opening and a second opening. A sealing membrane expands across the first opening, whereby a lid is formed. The lid is a flexible soft membrane, optionally provided with an opening in the centre, or off-set the centre (eccentric). When applied, the penis is inserted into the cup through the hole, which is slightly smaller than penis. Thereby, a lip seal is formed, wherein the membrane applies a small pressure on the skin and leakage of urine is avoided. This is believed to provide a comfortable and fluid-tight seal against the skin of the penis. The membrane material is flexible in order to secure a tight seal regardless of the variations in the patient's penis size and without compromising skin integrity. The primary urine collection device possesses an outlet-funnel at the bottom of the cup. Said outlet funnel can be provided with a connector with a sufficient bore to cope with the urine flow from the cup to e.g. a secondary urine collection device, comprising e.g. a bag or bladder-like structure.

According to an embodiment of the invention, said sealing membrane is adapted to cause minimal impact and/or tissue damage or discomfort for the user, such as providing a pressure of less than 25 mBar on the skin of the penis. Without wanted to be bound by any theory, it is believed that a pressure of more than 25 mBar can cause tissue damage, such as cell necrosis and pressure sores.

According to another embodiment, the lip seal provides a pressure of less than 50, 45, 40, 35, 30, 25, 20, 15, 10 or less than 10 mBar, such as 5 mBar on the skin of the penis. It is understood, that the penis diameter can vary, and that said pressure indications are related to a flaccid/relaxed/minimal diameter state of the penis. The pressure indications can also be average measures, such as a 6, 12, 24 or 48 hours average. According to a further embodiment of the invention, the membrane is of sufficient flexibility, to provide a minimal increase in pressure on the skin of the penis during e.g. erection. Such an increase in pressure can e.g. be less than 100, 80, 60, 50, 45, 40, 35, 30, 25, 20, 15, 10, or even 5 mBar. According to yet another embodiment, the sealing membrane is of such a flexibility that the lip seal essentially does not block, limit and/or reduces blood flow, and thus not cause priapism-related issues or problems.

It is understood that a cross section of a penis is not a perfect circle, and not perfectly symmetrical. According to an embodiment of the invention, the sealing membrane of the urine collection system comprises an essentially circular opening slightly smaller than the circumference of the penis shaft. Said opening can be placed in the centre or in an eccentric position, and the opening can typically have a diameter of e.g. 15-40 mm, or 23-33 mm. According to another embodiment of the invention, the circumference of the opening can e.g. be 1, 2, 3, 4, 5, 6, 7, 8, 9, 10, 12, 14, 16, 18 or 20 mm smaller than the circumference of the user's penis shaft. However, the (average) radius of said cross section can e.g. be 0.1-1, 1-2, 2-3, 3-4, 4-5, or more than 5 mm smaller than the radius of the opening in the sealing membrane.

According to an embodiment of the invention, the sealing membrane comprises an opening of organic shape, an elongated opening, or an elliptic opening. According to another embodiment, the circumference of the opening of more organic shape, elongated shape, or elliptic shape can be e.g. 1, 2, 3, 4, 5, 6, 7, 8, 9, 10, 12, 14, 16, 18 or 20 mm smaller than the circumference of the user's penis shaft. Usually, the maximum extend of said opening is smaller than the diameter of the penis.

According to an embodiment of the invention, the sealing membrane is provided with guide means enabling the user to provide an appropriate opening. Suitable guiding means can e.g. comprise concentric circles or essentially concentric organic shapes imprinted or visualized on the sealing membrane. An advantage can be that a user can provide an appropriately-sized opening in the membrane, e.g. by cutting or carving. It can also be advantageous that a user does not have to provide details about penis size when e.g. buying or ordering a device according to the invention, which can be perceived as shameful. A further advantage can be that the logistic requirements for keeping different sizes on stock, e.g. at a pharmacy, nursery home, hospital, or even supermarket can be reduced and/or minimized.

According to an embodiment of the invention, the sealing membrane has a thickness of 1 mm or less, e.g. a thickness between 0.75-1.0; 0.50-0.75; 0.25-0.50; 0.30-0.80; 0.10-0.25; or 0.05-0.10 mm; or a thickness around 0.1, 0.2, 0.3, 0.35, 0.4, 0.45, 0.5, or 0.6 mm. Said thickness can e.g. be measured essentially at, or 1-2 mm away from, the opening of the membrane. According to a further embodiment, the membrane has different thicknesses at different areas/portions of the membrane, such as thinner at or near the opening, and thicker and/or stronger further away from said opening, e.g. to provide membrane attachment means to attach the membrane around a primary opening of the primary urine collection device. There, the membrane can be thicker, such as more than 0.75-1.0 mm, 0.50-0.75 mm, 0.25-0.50 mm, 0.10-0.25, or 0.05-0.10 mm, or more than 0.1, 0.2, 0.3, 0.35, 0.4, 0.45, 0.5, 0.6, 0.7, 0.8, 0.9, or 1 mm. Without wanted to be bound by any theory, it is believed that the thickness of the membrane, optionally combined with selection of the appropriate membrane material(s), can be critical to proper functioning.

According to an embodiment of the invention, the sealing membrane comprises and/or is made of a polymer material, such as a material selected from the group consisting of polymer(s), thermoplastic elastomer(s), styrenic block polymer(s), polyolefin blend(s) (TPOs), elastomeric alloy(s), thermoplastic polyurethane(s) (TPUs), thermoplastic co-polyester(s), thermoplastic polyamide(s), licones(s), TPE's, hydrogels, natural rubber, and/or latex. The sealing membrane can also essentially (such as more than 80%, 85%, 90%, 95%, 97%, 98%, 99%, 99.5% or more) be made of a material selected from the group consisting of polymer(s), thermoplastic elastomer(s), styrenic block polymer(s), polyolefin blend(s) (TPOs), elastomeric alloy(s), thermoplastic polyurethane(s) (TPUs), thermoplastic co-polyester(s), thermoplastic polyamide(s), silicones(s), TPE's, hydrogels, natural rubber, and/or latex. According to a further embodiment of the invention, different sections/portions of said membrane can comprise two or more different materials, such as two or more compatible materials selected from a material selected from the group consisting of polymer(s), thermoplastic elastomer(s), styrenic block polymers), polyolefin blend(s) (TPOs), elastomeric alloy(s), thermoplastic poly-urethane(s) (TPUs), thermoplastic co-polyester(s), thermoplastic polyamide(s), and silicones(s), including any combination(s) thereof. According to another embodiment of the invention, the choice of choice of materials for the sealing membrane comprises TPE's, hydrogels, natural rubber, or latex.

According to an embodiment of the invention, the material of the sealing membrane has a shore A hardness of less than 30 or 25. According to another embodiment, the material of the sealing membrane has a shore A hardness of less than 50, 45, 40, 35, 30, 25, 20, 15, 10. Said shore A hardness of the sealing membrane can be provided at or near the opening, such as 1-2 mm away from the opening. According to another embodiment of the invention, said sealing membrane has a different shore A hardness at different sections/portions of said membrane, e.g. due to different membrane thicknesses and/or use of different materials and/or compositions.

According to an embodiment of the invention, the primary urine collection device and the sealing membrane are provided as a single integrated part. According to another embodiment, the primary urine collection device and the sealing membrane are and/or comprise two or more parts.

According to an embodiment of the invention, the primary urine collection device comprises sealing membrane attachment means providing a releasable attachment of the sealing membrane to the primary urine collection device. Said sealing membrane attachment means can be a separate element, releasably connectable to e.g. the cup and the membrane, such as an elastic, flexible structure, such as an elastic rubber ring, dimensioned to provide an essentially fluid-tight and releasable attachment of the membrane to the cup, whereby the first opening is covered (apart from the opening in the membrane, of course). According to another embodiment of the invention, the sealing membrane attachment means are incorporated and non-releasably provided as an integrated feature of the sealing membrane.

An embodiment of a sealing membrane 110 with membrane attachment means 120 is shown in FIG. 8.

According to an embodiment of the invention, the sealing membrane attachment means is/are re-usable. According to another embodiment, the sealing membrane attachment means is/are intended for single use. In a further embodiment, the sealing membrane attachment means is/are re-usable to a lesser extent, such as 2-5, or 5-10 times re-usable.

According to an embodiment of the invention, the sealing membrane attachment means comprises one or more reinforcement means, optionally comprising a circular or essentially circular ring structure. Said reinforcement can be of other shapes, such as organic, elliptic, egg-shaped, and the like, or even square or rectangular, or any combination of rounded and straight sections. Often, but not necessarily, the shape of the reinforcement will mimic the shape of the first opening of the usually cup/primary urine collection device. Although shown circular and symmetrical, the cross section of the first opening can be of other shape, such as organic, elliptic, egg-shaped, and the like, or even square or rectangular, or any combination of rounded and straight sections.

The cup or urinal of the primary urine collection device can e.g. be moulded in rigid, semi rigid or soft materials. Examples of materials comprise PVC, PU, PP, TPE's, hydrogels, natural rubber, latex and/or silicone. Moulding techniques can e.g. be injection moulding or blow moulding. The shape and size may be less critical, as long as the cup reservoir or volume is large enough to receive the urine during micturition without overflow, but not so big that discretion is compromised.

The cup and membrane lid can be manufactured in separate parts in order to use different materials, as the preferred material for the lid should be soft and elastic while the cup preferably should be more rigid. In another embodiment of this invention, the cup and lid could be manufactured in one operation, such as e.g. by blow moulding, rotation moulding, dipping process, or the injection-extrusion-blow moulding techniques used e.g. for the manufacture of toothpaste tubes.

The outlet in the bottom of the cup, also termed outlet funnel, is provided with a bore or diameter sufficiently large to allow a flow similar to, or larger than micturition flow rates. Said outlet funnel can be mounted at an angle to the cup in order to secure a free flow, e.g. when the patient is using the urinal while sitting down. Likewise, the outlet funnel can be pointing to the left or to the right, instead of in the centre. This is believed to add to user comfort, as men's testicles are not symmetrical, whereby either a “left” or “right” outlet funnel will be conceived as more comfortable by the user.

According to an embodiment of the invention, the urine collection system comprises fastening means adapted to provide a releasable connection between the first urine collection device and a garment worn by said user, such as underpants, drawers and/or shorts. Such fastening means may comprise adhesive means, and/or hooks and loops such as Velcro. Suitably, such adhesive means may comprise to matching adhesive Velcro strips, wherein one of the strips is attached on the inside of the user's underwear, and the corre-sponding second strip is attached on an outer surface of the cup (see e.g. FIG. 4). The adhesive means assist and/or provide a suitable positioning of the primary urine collection system. It is understood, that loose garments, such as boxer shorts could be less suitable, or even unsuitable, and that tighter fitting shorts would be more suitable. According to a further embodiment, underpants which ensure that the cup stays in place on penis may increase the comfort during wear. This may require the use of e.g. a strip of fastening means such as Velcro, adhesive tape or the like fixed or mounted on the ridge of the cup, such that it attaches to another (Velcro) part, which is fixed to the underpants perpendicular to the first. Thereby, fixation and adjustment can be achieved in an easy and secure/reliable fashion. This may enable the patient or user to adjust and/or fix the cup in any position needed from the size and shape of his penis and his underpants of choice.

In an embodiment, an opening is provided in the underpants, such as a circular opening or a buttonhole sewn, with a size and position suitable to lead a connection means tubing from the primary urine collection device to the secondary urine collection device. In another embodiment, said opening in the underpants assists in securing the primary urine collection device in the desired position in order to avoid leakage. Furthermore, said opening in the underpants may assist in securing a free flow of urine from the primary to the secondary urine collection device.

In an embodiment a fly in the underpants could be sufficient large to allow placement of the primary cup through the fly. This is needed if the user prefers to connect the system before mounting on the leg and penis. The fly should be of a design allowing correct and secure positioning of the cup and guidance of the tube to be placed in a kink-free position.

According to an embodiment of the invention, the primary urine collection device comprises an outflow funnel, said outflow funnel being provided at an angle α of between 0-90, 5-80, 10-60, 15-50, 25-45, 20-40, or 25-35 degrees measured against the sealing membrane. Further embodiments are e.g. visualized in FIG. 5-7. Providing the outflow funnel at an angle α may assist in avoid kinking and thereby avoiding block of the outflow. FIG. 5 shows an embodiment of a primary urine collection device 100, wherein the outflow funnel 160 is provided at an angle α of e.g. 10-60 degrees measured against the plane of the sealing membrane 110. FIG. 6 shows a drawing of an embodiment of a primary urine collection device 100 comprising a sealing membrane 110 with an opening 112, re-enforcement means 115, and an outflow funnel 160.

According to an embodiment of the invention, the primary urine collection device may not be vertically symmetric around the centre axis, and the outlet funnel can be provided at an angle of 1-45, 5-30, 10-20, 12-16, or around 14 degrees to the left or to the right, wherein said angle β measured against the primary devices centerline. An embodiment of a primary urine collection device 100, wherein the outflow funnel 160 is provided non-symmetrically with respect to the axis A-A, such as pointing to the left (user's perspective) is shown in FIG. 7, with an angle β of around 14 degrees (left).

According to a first aspect of the invention, a secondary urine collection device is provided, said secondary urine collection device being adapted to accommodate body fluids such as urine.

According to an embodiment of the invention, the secondary urine collection device comprises a bladder-like structure adapted to collect body fluids such as urine, and integrated means to secure said secondary urine collection device to a leg of the user. The secondary urine collection device may thus be forming a legging or gaiter.

According to an embodiment of the invention, the bladder-like structure comprises spacing means. Said spacing means are adapted to avoid collapsing of the walls of the bladder-like structure, in order to facilitate, improve, and/or increase the rate of filling of the bladder. Without wanting to be bound by any theory, it is believed that said spacing means may also avoid or reduce back-log of urine in the primary urine collection device and/or the connection means. According to an embodiment of the invention, spacing means could be provided by 3 dimensional forming of the outer layer film, such as e.g. illustrated in FIGS. 11A+B. Forming of the outer layer can e.g. be provided by standard thermoforming manufacturing processes, such as e.g. by vacuumforming or pressing.

According to an embodiment of the invention, the securing means of the secondary urine collection device comprise one or more elastic tubular structure(s) adapted to encompass at least a part of the leg of the user forming a legging. Several embodiments of secondary urine collection device 200 comprise e.g. one or more elastic tubular structure(s) are presented in FIG. 9 A-E. The elastic tubular structure may comprise e.g. one or more of elastic fabric, sports stretch, rib stretch, elastic film, elastic nonwoven, elastic net, or other materials are known in the art. Suitable fabrics comprise also elastic type fabrics such as “sportsstrech”, and/or fabrics used for tights or compression stockings. According to another embodiment, Velcro tape is added as an extra security for the patient to prevent the bag from sliding down when full. In another embodiment, the fixation may comprise elastic tape sewn or welded to top and bottom of the bag (e.g. FIG. 9C). Optionally, the elastic material can be supplemented by a Velcro closing mechanism as needed.

According to an embodiment of the invention, the elastic tubular structure and the bladder-like structure are secured and/or joined by one or more of welding, glue, adhesive, sewing, and/or other means commonly known in the art. Thus a urine bag or bladder-like structure adapted for fixation to the leg can be an integrated part of the secondary urine collection device. This may comprise use of means such as Velcro tape fasteners or by designing the urine bag essentially as a cylinder or tube which fits the leg (FIG. 9 A-E).

According to another embodiment, the bladder-like structure can be encompassed at least in part in a pocket or the like of a stocking leg-like structure. In a further embodiment, the bladder-like structure is essentially encompassed, such as more than e.g. 80%, 90%, 95%, or 99%, or completely encompassed in a pocket or the like of said stocking leg-like structure. The bladder-like structure can also be loosely inserted inside a fixing holster or sock adapted to be mounted or fixated around the calf of the leg.

A secondary urine collection system according to the invention can either be symmetrical, or non-symmetrical, such as adapted for a left or a right leg of a user. An embodiment of a non-symmetrical secondary urine collection device is e.g. shown in FIG. 9E. Apart from securing means 220, orientation indication means 210, 215 can be provided, adapted to assist the user in preferably intuitively applying the secondary urine collection device. These orientation indication means 210, 215 may comprise an inscription, assisting in correct orientation top to bottom, as well as inside out. Furthermore, a label, such as a label similar to labels commonly found in the neck area of clothing such as T-shirts or shirts, assisting in finding the appropriate orientation in terms of front and back.

A further aspect of the invention pertains to connection means adapted to provide a leakage-free connection between a primary urine collection device according to the second aspect, and a secondary urine collection device according to the first aspect. According to an embodiment of the invention, the connection means comprise a flexible tube or tubing, such as tubes/tubings that are known in the art for connecting a primary urine collection device with secondary urine collection device(s).

According to an embodiment, the urine collection system according to the invention comprises a primary and a secondary urine collection device, and connection means adapted to provide a leakage-free connection between said primary urine collection device, and said secondary urine collection device. Optionally, according to another embodiment, said urine collections system may comprise one or more flow controlling means such as one or more, valve, tap, and/or contra-valve, non-return valve, and any combination thereof. A function of said flow controlling means is avoidance and/or reduction of unintended spilling body fluids such as urine under use, wherein said use comprises emptying urine collected in the secondary urine collection device, or replacement or cleaning of any one of primary urine collection device, secondary urine collection device, and/or connection means. According to a further embodiment, said contra valve(s) and/or non-return valve(s) allow only for unidirectional flow of bodyfluids/urine, such as only in the direction of primary urine collection device to the secondary urine collection device. Said flow controlling means can be located or comprised in any one of primary urine collection device, secondary urine collection device, and/or connection means.

According to an embodiment of the invention, the urine collection system comprises noise abatement means adapted to reduce noise during micturition. Said noise abatement means can be located or comprised in any one of primary urine collection device, secondary urine collection device, and/or connection means, including any combination thereof.

According to an embodiment, the urine collection system according to the invention may comprise anti-kinking means. Thus, the primary urine collection device, the secondary urine collection device, and/or connection means and any combination thereof may comprise anti-kinking means adapted to avoid and/or prevent kinking of the urine collection system or one or more parts thereof. A function of the anti-kinking means is to avoid back-log of body fluids in any one of primary urine collection device, secondary urine collection device, and connection means, and any combination thereof.

A further aspect of the invention relates to diameter determination means adapted to assess a diameter of a body part, such as a penis or a leg, such as the calf of a leg. According to one embodiment of the invention, said diameter determination means comprise(s) a ribbon-like structure provided with a symbol- and/or colour-code for rapid and/or simple assessment and/or selection of the appropriate sealing membrane opening, and/or diameter/size of the secondary urine collection device. An embodiment of diameter determination means is e.g. shown in FIG. 10, where the diameter determination means (400) may comprise a ribbon-like structure (410) provided with a symbol and/or color- or grey-scale code (420), and assessment means (430).

Diameter determination means may comprise(s) a ribbon-like structure provided with a symbol- and/colour-code for assessment and/or selection of the appropriate sealing membrane opening, and/or diameter/size of the secondary urine collection device.

According to a further aspect, a kit is provided comprising a primary urine collection device according to the second aspect, and a secondary urine collection device according to the first aspect. In an embodiment of the invention, said kit may comprise connection means according to one of the further aspects. According to another embodiment, said kit may further comprise penis and or leg/calf diameter determination means, such as diameter determination means according to one of the further aspects.

A further aspect of the invention relates to accommodation means, such as accommodation means for the primary urine collection device and/or the connection means. According to an embodiment of the invention, such accommodation means may comprise e.g. an opening, such as a slot, fly, or button hole in a garment, such as an underwear, wherein said opening is adapted to provide passage of the primary collection device or at least a part or portion of the connection means, such as a tubing or the like from the primary urine collection device to a secondary urine collection device.

A further aspect of the invention concerns anti-kinking means, adapted to avoid a reduction and/or blockage of flow of body fluids from the primary urine collection device to the secondary urine collection device. According to an embodiment of the invention, anti-kinking means comprise the position of the outflow funnel in relation to the sealing membrane, optionally in combination with accommodation means according to one of the further aspects. According to another embodiment, said anti-kinking means comprise e.g. selection of a suitable material, such as PVC, silicone or polyurethane, or using a corrugated tubing system. In an embodiment of the invention, the cup/primary urine collection device is provided with hinging means, adapted and/or designed to provide movement of the funnel, such as up and down, optionally combined with a sidewards (left-right) movement, thus transferring and or absorbing the user's movement(s) without kinking the tube, and/or funnel.

According to an embodiment, the hinge means or hinge is made by reducing material thickness of the cup in an area around the base of the funnel.

A further aspect concerns a modification, enabling the use of the primary urine collection system as chamber pot at night or if the patient is bedridden. Thus, in a night version, the bag or secondary urine collection device is adapted to e.g. be lying on the floor, or hung on a hanger of known design. According to an embodiment of the invention, the secondary urine collection device comprises hanging means, such as a loop (see e.g. FIG. 9B), adapted for hanging the secondary urine collection device onto said hanger. In a further embodiment, said loop is also adapted to putting on or strapping on the secondary urine collection device. In yet another embodiment, said loop may also comprise orientation indication means.

### EXAMPLES

### Example 1

According to an embodiment, a body fluid/urine a collection system for men is provided, comprising a urinal or cup with means of fixation in the underpants and a urine leg bag for the storage of the void. The urinal and the bag are connected by means of tubing. In this embodiment/example, the sealing between penis and urinal is established and maintained by means of a lip-seal structure constructed as a membrane with a hole slightly smaller than penis circumference. Fixation of the urinal in the correct or appropriate position can be provided by means of e.g. Velcro (or Veicro like products). The urine bag adapted for fixation to the leg of the user can be an integrated part of the bag. Suitably, the sealing membrane delivers a pressure on the penis skin of less than 25 mBar. Such a membrane can e.g. be made of a polymer such as silicone or a TPE with a shore(A) hardness of less than 25, and may comprise or consist of a hydrogel. The membrane may posses a thickness of less than 1 mm. Optionally, the user or customer produces the hole in the membrane prior to use, if the device is provided without hole, or the hole is too small. Said membrane and the urinal are manufactured as separate parts; alternatively they can be produced in one shot (as one unit). With respect to fixation of the urinal in an appropriate position, such as by the use of Velcro or the like, fixation could also be achieved by means of adhesives between the urinal and the underpants or between urinal and underpants through an intermediary medium. The cup can be provided with an outflow funnel at an angle of between 25-35 degrees measured against the flat backside of the cup. With respect to the urine bag, fixation to the leg can be accomplished e.g. by the use of joining elastic fabric (sports stretch, rib stretch), elastic film, an elastic nonwoven or an elastic net to the urine bag thus forming a tube that fits the calf of the leg. Joining of the urine bag and the elastic part can be achieved by e.g. sewing, welding or by means of adhesive, such as glue or the like. Fixation to the leg can be achieved by the use of several, such as two or more elastic belts crossing the calf thus holding the bag in place. Alternatively, or in combination, fixation to the leg can be achieved by one or more ribbons (made by elastic tape or Velcro or similar products as Velcro) or the like crossing the calf thus holding the bag in place. Furthermore, the bag can be an integrated part of a stocking leg. The bag may then be mounted in the inside or the outside of such stocking. The mounting can e.g. comprise sewing, welding or adhesive means such as gluing.

1. A secondary urine collection device comprising a bladder-like structure adapted to collect body fluids such as urine, and integrated securing means adapted to secure the secondary urine collection device to a leg of the user.

2. A secondary urine collection device according to claim 1, wherein the securing means comprise one or more elastic tubular structure(s) adapted to encompass at least a part of the leg of the user.

3. A secondary urine collection device according to claim 2, wherein the elastic tubular structure comprises one or more of elastic fabric, sports stretch, rib stretch, elastic film, elastic nonwoven, elastic net, elastic tape and velcro.

4. A secondary urine collection device according to claim 3, wherein the elastic tubular structure and the bladder-like structure are secured and/or joined by one or more of welding, glue, adhesive, and/or sewing.

5. A secondary urine collection device according to claim 1, wherein the bladder-like structure is encompassed at least in part in a pocket of a stocking leg-like structure.

6. A secondary urine collection device according to claim 1 further comprising noise abatement means adapted to reduce noise during micturition.

7. A urine collection system for a male user, said collection system comprising a primary urine collection device, wherein the primary urine collection device is adapted to accommodate at least a glans-comprising portion of a penis, said primary urine collection device comprising sealing means adapted to provide an essentially fluid-tight connection between the primary urine collection device and the penis, and wherein said essentially fluid-tight connection comprises a sealing membrane provided with an opening comprising a lip-seal structure sealing against the skin of the penis.

8. A urine collection system according to claim 7, wherein the sealing membrane is adapted to provide a pressure of less than 25 mBar on the skin of the penis.

9. A urine collection system according to claim 7, wherein the sealing membrane comprises an essentially circular opening slightly smaller than the circumference of the penis shaft.

10. A urine collection system according to claim 7, wherein the sealing membrane comprises an opening of organic shape, an elongated opening, or an elliptic opening.

11. A urine collection system according to claim 7, wherein the sealing membrane is provided with guide means enabling the user to provide an appropriate opening.

12. A urine collection system according to claim 7, wherein the sealing membrane has a thickness of 1 mm or less, a thickness between 0.75-1.0 mm, 0.50-0.75 mm, 0.25-0.50 mm, 0.10-0.25, or 0.05-0.10 mm, preferably 0.2-0.3 mm or a thickness around 0.2, 0.3, 0.4, 0.5, or 0.6 mm.

13. A urine collection system according to claim 7, wherein the sealing membrane comprises and/or is made of a polymer material, such as a material selected from the group consisting of polymer(s), thermoplastic elastomer(s), styrenic block polymer(s), polyolefin blend(s) (TPOs), elastomeric alloy(s), thermoplastic polyurethane(s) (TPUs), thermoplastic co-polyester(s), thermoplastic polyamide(s), natural rubber latex and silicones(s).

14. A urine collection system according to claim 7, wherein the material of the sealing membrane has a shore A hardness of approximately 30, less than 30 or 25.

15. A urine collection system according to claim 7, wherein the primary urine collection device and the sealing membrane are provided as a single integrated part.

16. A urine collection system according to claim 7, wherein the primary urine collection device and the sealing membrane are two or more parts.

17. A urine collection system according to claim 16, wherein the primary urine collection device comprises sealing membrane attachment means providing a releasable attachment of the sealing membrane to the primary urine collection device.

18. A urine collection system according to claim 17, wherein the sealing membrane attachment means is/are re-usable.

19. A urine collection system according to claim 17, wherein sealing membrane attachment means comprises one or more reinforcement means, optionally comprising a circular or essentially circular ring structure.

20. A urine collection system according to claim 7, wherein the urine collection system comprises fastening means adapted to provide a releasable connection between the primary urine collection device and a garment worn by said user.

21. A urine collection system according to claim 20, wherein the fastening means comprise(s) adhesive means, and/or hooks and loops such as Velcro.

22. A urine collection system according to claim 7, wherein the urine collection system comprises a secondary urine collection device, and connection means adapted to provide a leakage-free connection between said primary urine collection device and said secondary urine collection device, and optionally one or more flow controlling means such as one or more valve, tap, and/or contra-valve, and any combination thereof.

23. A urine collection system according to claim 7, wherein the primary urine collection device comprises noise abatement means adapted to reduce noise during micturition.

24. A urine collection system according to any of the claims claim 7, wherein the primary urine collection device, the secondary urine collection device, and/or connection means comprise anti-kinking means adapted to avoid and/or prevent kinking of the urine collection system or one or more parts thereof.

25. A urine collection system according to claim 7, wherein the primary urine collection device comprises an outflow funnel, said outflow funnel being provided at an angle α of between 10-60, 15-50, 25-45, 20-40, or 25-35 degrees measured against the sealing membrane.

26. A urine collection system according to claim 7, wherein the secondary urine collection device comprises a bladder-like structure adapted to collect body fluids such as urine, and integrated securing means adapted to secure said secondary urine collection device to a leg of the user.

27. A urine collection system according to claim 26, wherein the securing means comprise one or more elastic tubular structure(s) adapted to encompass at least a part of the leg of the user.

28. A urine collection system according to claim 27, wherein the elastic tubular structure comprises one or more of elastic fabric, sports stretch, rib stretch, elastic film, elastic nonwoven, elastic net, elastic tape and velcro.

29. A urine collection system according to claim 28, wherein the elastic tubular structure and the bladder-like structure are secured and/or joined by one or more of welding, glue, adhesive, and/or sewing.

30. A urine collection system according to claim 21, wherein the bladder-like structure is encompassed at least in part in a pocket of a stocking leg-like structure.

31. A secondary urine collection device according to claim 26.

32. A kit comprising a primary urine collection device according to claim 7, and a secondary urine collection device wherein the secondary urine collection device comprises a bladder-like structure adapted to collect body fluids such as urine, and integrated securing means adapted to secure said secondary urine collection device to a leg of the user.

33. A kit according to claim 32, further comprising penis and/or leg/calf diameter determination means.

34. A kit according to claim 33, wherein the diameter determination means comprise(s) a ribbon-like structure provided with a symbol- and/or colour-code for assessment and/or selection of the appropriate sealing membrane opening, and/or diameter/size of the secondary urine collection device.

35. A urine collection device for a male user, said collection device being adapted to accommodate at least a glans-comprising portion of a penis, said urine collection device comprising sealing means adapted to provide an essentially fluid-tight connection between the first urine collection device and the penis, and wherein said essentially fluid-tight connection comprises a sealing membrane provided with an opening comprising a lip-seal structure sealing against the skin of the penis.
